(19) United States
(12) Patent Application Publication
(10) Pub. No.: US 2010/0311765 A1
(43) Pub. Date: Dec. 9, 2010

(54) METABOLIC DEGRADATION INHIBITORS FOR ANTI-HYPERPROLIFERATIVE AGENTS

(76) Inventors: Barry James Maurer, Idalou, TX (US); Charles Patrick Reynolds, Lubbock, TX (US)

Correspondence Address: MYERS BIGEL SIBLEY & SAJOVEC, PO BOX 37428, RALEIGH, NC, 27627, US

(21) Appl. No.: 12/679,795

(22) PCT Filed: Sep. 26, 2008
(86) PCT No.: PCT/US2008/011207

§ 371 Date: Jul. 12, 2010

Related U.S. Application Data
(60) Provisional application No. 60/975,950, filed on Sep. 28, 2007.

(51) Int. Cl. A61K 31/4164; A61K 31/496; A61K 31/4196; A61K 31/16; A61P 35/04

(52) U.S. Cl. 514/254.07; 514/396; 514/383; 514/613

(57) ABSTRACT

The present invention provides methods of increasing an amount of a treatment agent in the body, a cancer or tumor. The methods include administering an inhibitor of the metabolic degradation or conversion of the treatment agent to a subject undergoing treatment for a hyperproliferative disorder with said treatment agent. Methods of treating hyperproliferative disorders, tumors and cancers are also provided.

### RELATED APPLICATION DATA

This application claims priority to and the benefit of U.S. Patent Application Ser. No. 60/975,950, filed Sep. 28, 2007, the disclosure of which is incorporated by reference herein in its entirety.

### FIELD OF INVENTION

The present invention concerns combinations of metabolic degradation inhibitors and agents used for the treatment of hyperproliferative disorders, including cancers and tumors.

### BACKGROUND OF THE INVENTION

In the treatment of hyperproliferative disorders, including a cancer or tumor, the amount of an agent used to treat the disorder is usually limited by the side effects induced in the subject by the treatment agent(s). Moreover, for certain treatment agents, the amount of the agent used to treat the condition in the subject may be limited by the bioavailability of the agent when delivered using various delivery methods. Such delivery methods can include, but are not limited to, delivery by oral, buccal, intravenous, intraperitoneal or cutaneous administration. Accordingly, it is desirable to provide options that allow a desired amount of a therapeutic agent to be delivered to a subject.

### SUMMARY OF THE INVENTION

Embodiments of the present invention provide methods of increasing in the body an amount of a treatment agent, said method comprising administering an inhibitor of the metabolic degradation or conversion of the treatment agent to a subject undergoing treatment for a hyperproliferative disorder with said treatment agent.

Embodiments of the present invention further provide methods of treating a cancer or tumor comprising administering an inhibitor of the metabolic degradation or conversion of a treatment agent intended to treat the cancer or tumor, wherein said inhibitor increases the amount of the intended treatment agent in the body.

Embodiments of the present invention also provide methods of increasing in a cancer or a tumor the amount of an agent intended to treat the cancer or tumor comprising administering to a subject in need of treatment (a) the intended treatment agent, and (b) an inhibitor of the metabolic degradation or conversion of the intended treatment agent.

The present invention further concerns the use of an inhibitor as described herein for the preparation of a medicament or pharmaceutical formulation for carrying out a method as described herein.

### BRIEF DESCRIPTION OF THE DRAWINGS

FIG. 1 presents a graph showing results of the effect of ketoconazole on plasma fenretinide levels in nude mice.

FIG. 2 presents a graph showing results of the effect of ketoconazole on plasma fenretinide levels in nude mice (A), and the effect of ketoconazole on fenretinide levels in mouse liver.

FIG. 3 presents a graph showing results of the effect of ketoconazole on plasma fenretinide levels in NOD/SCID mice.

FIG. 4 presents a graph showing results of the effect of ketoconazole on fenretinide levels in human tumor xenograft tissue grown in nude mice.

FIG. 5 presents a graph showing results of the effect of other inhibitors on fenretinide plasma levels.

### DETAILED DESCRIPTION

The foregoing and other aspects of the present invention will now be described in more detail with respect to embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention pertains. The terminology used in the description of the invention herein is for the purpose of describing particular embodiments only and is not intended to be limiting of the invention. As used in the description of the invention and the claims set forth herein, the singular forms “a,” “an,” and “the” are intended to include the plural forms as well, unless the context clearly indicates otherwise.

The present invention relates to the discovery that the amount of a treatment agent(s) that can be delivered to a patient, or in the case of a cancer, the amount of the treatment agent(s) delivered to the cancer, can be increased by co-treating the subject with an inhibitor that decreases the metabolism or conversion of the treatment agent(s) into less-active or non-active forms or excretable derivatives or other derivatives. The success of this approach for a given delivered amount of the treatment agent can be determined by measuring an increase of the treatment agent in the blood, or in the blood plasma, or in the tissues, or in the cancer, as compared to the amount of the agent so measured in the subject or cancer when the inhibitor is not used.

Accordingly, embodiments of the present invention provide methods of treating a hyperproliferative disorder, including a cancer, in a subject in need of such treatment, comprising administering to said subject a pharmaceutical combination containing an amount of: (a) an agent intended for the treatment of the disorder, or a pharmaceutically acceptable salt thereof, and an inhibitor of the metabolic degradation or conversion of the intended treatment agent, or a pharmaceutically acceptable salt thereof; (b) an anti-cancer agent, or a pharmaceutically acceptable salt thereof, and an inhibitor of the metabolic degradation or conversion of the anti-cancer agent; (c) a combination containing fenretinide (i.e., N-(4-hydrophenyl)retinamide, 4-HPR) and ketoconazole; or (d) a combination containing fenretinide (i.e., N-(4-hydrophenyl)retinamide, 4-HPR) and fluconazole.

The temporal relationship of the delivery of metabolic inhibitor to the delivery of the intended treatment agent can vary for any given inhibitor so long as the beneficial effect of increasing the level of the treatment agent in the body or in the cancer or tumor is achieved. In certain embodiments of the present invention, the metabolic inhibitor is delivered before, during and/or after the delivery of the intended treatment agent(s). In certain embodiments of the present invention, the metabolic inhibitor is delivered after the delivery of the intended treatment agent(s).

In certain embodiments of the present invention, the metabolic inhibitor is pharmaceutically compounded together with the treatment agent(s) for delivery. In other embodiments, the inhibitor and the treatment agent(s) are formulated for delivery separately.

In certain embodiments, the metabolic inhibitors may be inhibitors of hepatic or non-hepatic cytochrome P₄₅₀ enzymes. In other embodiments, the metabolic inhibitors may be inhibitors of hepatic or non-hepatic methyltransferases.

Anti-cancer agents that may be useful for the present invention include retinoids. Retinoids useful in the methods described herein include vitamin A derivatives. In particular, vitamin A derivatives include, but are not limited to, fenretinide (N-(4-hydrophenyl)retinamide, 4-HPR). Inhibitors of the metabolic degradation or conversion pathway for anti-cancer agents that may be used according to the present invention include, but are not limited to, imidazole derivatives and triazole derivatives. A particular retinoid relevant to the present invention is fenretinide. A particular imidazole metabolic inhibitor according to some embodiments of the present invention is ketoconazole. A triazole metabolic inhibitor according to some embodiments of the present invention is fluconazole.

A skilled practitioner will notice that the delivery of an amount of an intended treatment agent that may otherwise be insufficient to achieve a treatment effect against a specific hyperproliferative disorder, or cancer, may, by the method of the present invention, achieve a treatment effective amount of the agent in the body or in the cancer. This result is also an embodiment of the present invention.

It should be understood that a treatment effect against a hyperproliferative disorder or a cancer does not imply the cure of the hyperproliferative disorder or the cancer but does include any beneficial effect to the subject from such a treatment of a hyperproliferative disorder or cancer, including, but not limited to, the reduction or control of pain or discomfort, a reduction in the rate of growth of a cancer or tumor, a temporary cessation of growth of a cancer or tumor, a reduction in size of a cancer or tumor, or other beneficial effect.

Pharmaceutical formulations. Active compounds as used herein include both inhibitors as described herein and intended treatment agents as described herein.

The active compounds described above may be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% or 99% by weight of the active compound. One or more active compounds may be incorporated in the formulations of the invention, which may be prepared by any of the well known techniques of pharmacy comprising admixing the components, optionally including one or more accessory ingredients.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the active compound in a flavoured base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the active compound(s), which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising an active compound(s), or a salt thereof, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. Suitable formulations comprise citrate or bis\tris buffer (pH 6) or ethanol/water and contain from 0.1 to 0.2M active ingredient.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same may be incorporated into lipid vesicles. In such an instance, due to the water solubility of the compound or salt, the compound or salt will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer which forms the structure of the liposome. In either instance, the liposomes which are produced may be reduced in size, as through the use of standard sonication and homogenization techniques.

Of course, the liposomal formulations containing the compounds disclosed herein or salts thereof, may be lyophilized to produce a lyophilizate which may be reconstituted with a pharmaceutically acceptable carrier, such as water, to regenerate a liposomal suspension.

Other pharmaceutical compositions may be prepared from the water-insoluble compounds disclosed herein, or salts thereof, such as aqueous base emulsions. In such an instance, the composition will contain a sufficient amount of pharmaceutically acceptable emulsifying agent to emulsify the desired amount of the compound or salt thereof. Particularly useful emulsifying agents include phosphatidyl cholines, and lecithin.

In addition to active compound(s), the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

The present invention further concerns the use of an inhibitor as described herein for the preparation of a medicament or pharmaceutical formulation for carrying out a method as described herein.

Dosage and routes of administration. As noted above, the present invention provides pharmaceutical formulations comprising the active compounds (including the pharmaceutically acceptable salts thereof), in pharmaceutically acceptable carriers for oral, rectal, topical, buccal, parenteral, intramuscular, intradermal, or intravenous, and transdermal administration.

The therapeutically effective dosage of any specific compound, the use of which is in the scope of present invention, will vary somewhat from compound to compound, and subject to subject, and will depend upon the condition of the subject and the route of delivery. As a general proposition, a dosage from about 0.1 to about 50 mg/kg will have therapeutic efficacy, with all weights being calculated based upon the weight of the active compound, including the cases where a salt is employed. Toxicity concerns at the higher level may restrict intravenous dosages to a lower level such as up to about 10 mg/kg, with all weights being calculated based upon the weight of the active base, including the cases where a salt is employed. A dosage from about 10 mg/kg to about 50 mg/kg may be employed for oral administration. Typically, a dosage from about 0.5 mg/kg to 5 mg/kg may be employed for intramuscular injection.

Treatment. The present invention pertains to treatment of hyperproliferative disorders such as tumors, cancers, and neoplastic disorders, as well as premalignant and non-neoplastic or non-malignant hyperproliferative disorders. The hyperproliferative disorder such as a tumor, cancer, or neoplastic disorder, as well as premalignant and non-neoplastic or non-malignant hyperproliferative disorder may be present within a subject or may be the actual tissue or sample thereof.

Examples of tumors, cancers, and neoplastic tissue that can be treated by the present invention include, but are not limited to, malignant disorders such as breast cancers; osteosarcomas; angiosarcomas; fibrosarcomas and other sarcomas; leukemias; lymphomas; sinus tumors; ovarian, uretal, bladder, prostate and other genitourinary cancers; colon, esophageal and stomach cancers and other gastrointestinal cancers; lung cancers; myelomas; pancreatic cancers; liver cancers; kidney cancers; endocrine cancers; skin cancers; and brain or central and peripheral nervous (CNS) system tumors, malignant or benign, including gliomas and neuroblastomas.

Examples of premalignant and non-neoplastic or non-malignant hyperproliferative disorders include, but are not limited to, myelodysplastic disorders; cervical carcinoma-in-situ; familial intestinal polyposes such as Gardner syndrome; oral leukoplakias; histiocytoses; keloids; hemangiomas; hyperproliferative arterial stenosis, inflammatory arthritis; hyperkeratoses and papulosquamous eruptions including arthritis. Also included are viral induced hyperproliferative diseases such as warts and EBV induced disease (i.e., infectious mononucleosis), scar formation, and the like. The methods of treatment disclosed herein may be employed with any subject known or suspected of carrying or at risk of developing a hyperproliferative disorder as defined herein.

Subjects. Subjects suitable to be treated according to the present invention include, but are not limited to, avian and mammalian subjects, and are preferably mammalian. Mammals of the present invention include, but are not limited to, canines, felines, bovines, caprines, equines, ovines, porcines, rodents (e.g. rats and mice), lagomorphs, primates, humans, and the like, and mammals in utero. Any mammalian subject in need of being treated according to the present invention is suitable. Human subjects are preferred. Human subjects of both genders and at any stage of development (i.e., neonate, infant, juvenile, adolescent, adult) can be treated according to the present invention.

Illustrative avians according to the present invention include chickens, ducks, turkeys, geese, quail, pheasant, ratites (e.g., ostrich) and domesticated birds (e.g., parrots and canaries), and birds in ovo.

The present invention is primarily concerned with the treatment of human subjects, but the invention can also be carried out on animal subjects, particularly mammalian subjects such as mice, rats, dogs, cats, livestock and horses or on animal or human tissue samples for veterinary purposes or research purposes, and for drug screening and drug development purposes.

### Example 1

### Effect of Ketoconazole on Fenretinide Levels in Mouse Plasma

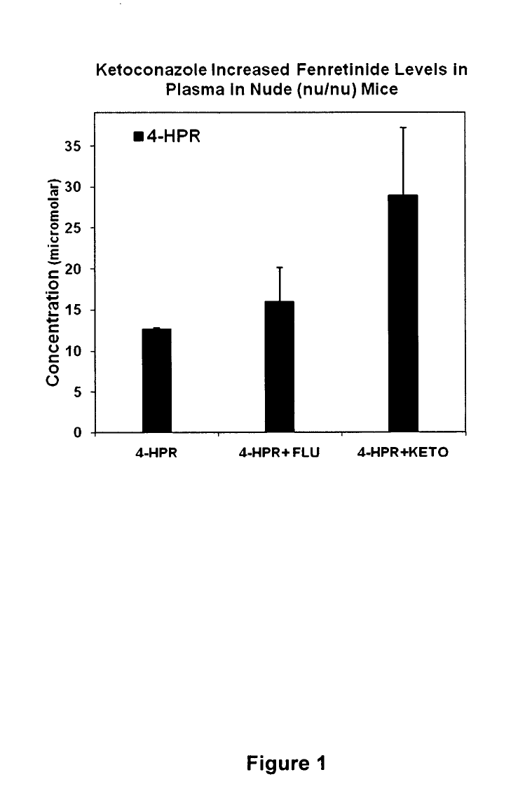

Ketoconazole increased fenretinide levels in mouse plasma to a greater extent than fluconazole. See FIG. 1. Fenretinide (i.e., N-(4-hydrophenyl)retinamide, 4-HPR) formulated for oral delivery in a vegetable oil (fatty acid) Lym-X-Sorb® matrix was delivered by gavage to nude (nu/nu) mice (n=3, per cohort) at 180 mg/kg/day, divided into two daily doses, for five doses (Control), or fenretinide was delivered together with fluconazole at 20 mg/kg/day, divided into two daily doses, for five doses (4-HPR+FLU), or fenretinide was delivered together with ketoconazole at 75 mg/kg/day, divided into two daily doses, for five doses (4-HPR+KETO). The fluconazole dose approximately corresponded to a human equivalent dose of 100 mg/day for a 70 kg human. The ketoconazole dose corresponded to a human equivalent dose of approximately 400 mg/day for a 70 kg human. These doses are within the standard range of human treatment doses for both of these antifungal agents. Animals were sacrificed four hours after the last drug dosing and levels of fenretinide measured in the plasma by a HPLC methodology. Results showed that ketoconazole significantly increased the mean plasma fenretinide levels from 12.6 micromolar with fenretinide-alone, (4-HPR), to 28.4 micromolar when ketoconazole (KETO) was co-administered with fenretinide, (P<0.04, one-sided t-test). In contrast, fluconazole (FLU) increased 4-HPR levels to a lesser extent (15.9 micromolar, P=0.15).

### Example 2

### Effect of Ketoconazole on Fenretinide Levels in Mouse Plasma and Liver Tissue

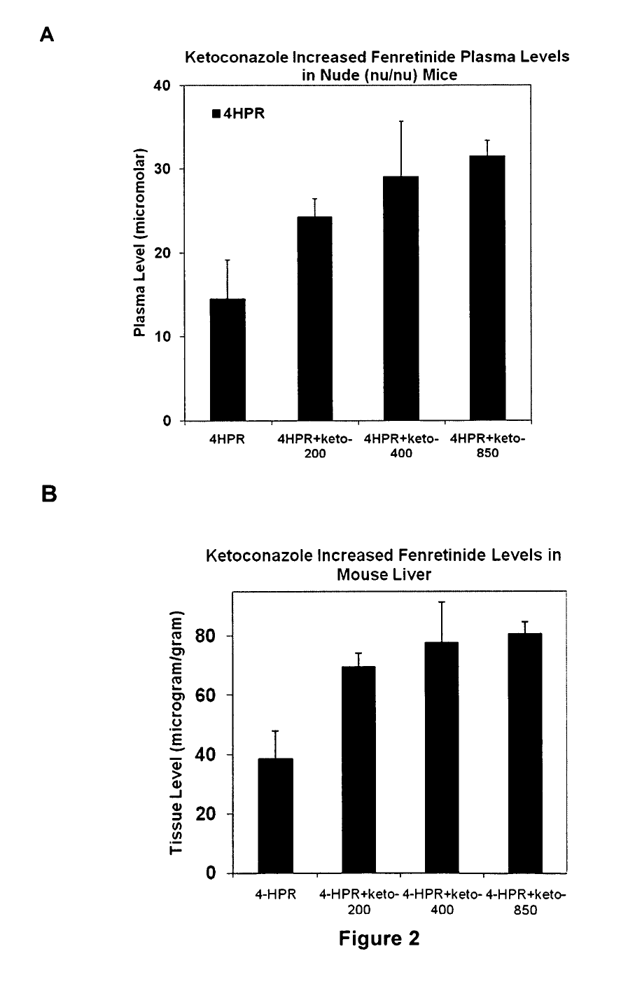

Ketoconazole increased fenretinide levels in mouse plasma and liver tissues. See FIG. 2. Fenretinide (i.e., N-(4-hydrophenyl)retinamide, 4-HPR) formulated for oral delivery in a vegetable oil (fatty acid) Lym-X-Sorb® matrix was delivered by gavage to nude (nu/nu) mice (n=3, per cohort) at 180 mg/kg/day, divided into two daily doses, for five doses total (Control), or alternatively, ketoconazole at doses of 37 or 75 or 150 mg/kg/day was delivered by gavage, divided into two daily doses, for nine total doses, starting two days prior to fenretinide at 180 mg/kg/day, divided into two daily doses, for five doses total (4-HPR+KETO). These ketoconazole doses approximately correspond to approximate human equivalent doses of 200, 400 and 850 mg per day for a 70 kg human, which are within the standard dose range for human treatment. Animals were sacrificed four hours after the last drug dosing and levels of fenretinide measured in the plasma and liver tissue by a HPLC methodology. Results showed that ketoconazole significantly increased the mean fenretinide level in both blood plasma (FIG. 2A), and liver tissue (FIG. 2B) at all ketoconazole dose levels tested compared to controls (P<0.05, for all comparisons, one-sided t-test). These results demonstrate that extended ketoconazole dosing can increase fenretinide plasma and tissue levels over a treatment course.

### Example 3

### Effect of Various Dosages of Ketoconazole on Fenretinide Levels in Mouse Plasma

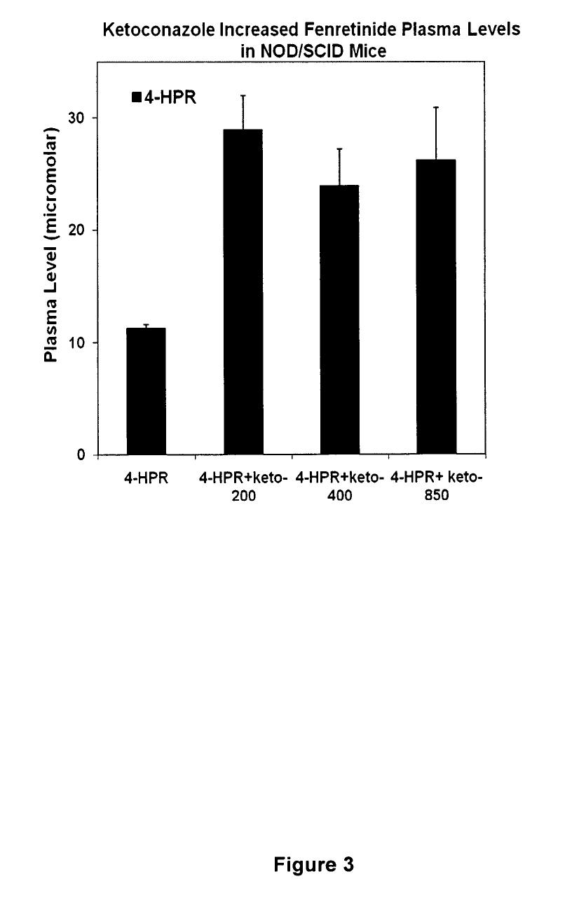

Ketoconazole increased fenretinide levels in mouse plasma. See FIG. 3. Fenretinide (i.e., N-(4-hydrophenyl)retinamide, 4-HPR) formulated for oral delivery in a vegetable oil (fatty acid) Lym-X-Sorb® matrix was delivered by gavage to NOD/SCID mice (n=3, per cohort) at 180 mg/kg/day, divided into two daily doses, for five doses total (Control), or alternatively, ketoconazole, at doses of 37 or 75 or 150 mg/kg/day, was delivered by gavage, divided into two daily doses, for nine total doses, starting two days prior to fenretinide at 180 mg/kg/day, divided into two daily doses, for five doses total (4-HPR+KETO). These ketoconazole doses approximately correspond to approximate human equivalent doses of 200, 400 and 850 mg per day for a 70 kg human, which are within the standard dose range for human treatment. Animals were sacrificed four hours after the last drug dosing and levels of fenretinide measured in the blood plasma by a HPLC methodology. Results showed that ketoconazole significantly increased the mean fenretinide level in plasma at all ketoconazole dose levels tested compared to controls (P<0.05, for all comparisons, one-sided t-test) in a second mouse strain. These data indicate that this effect is a generalized principle. These results demonstrate that prolonged ketoconazole dosing can increase fenretinide blood plasma levels over a treatment course.

### Example 4

### Effect of Ketoconazole on Fenretinide Levels in Human Tumor Xenograft Tissue

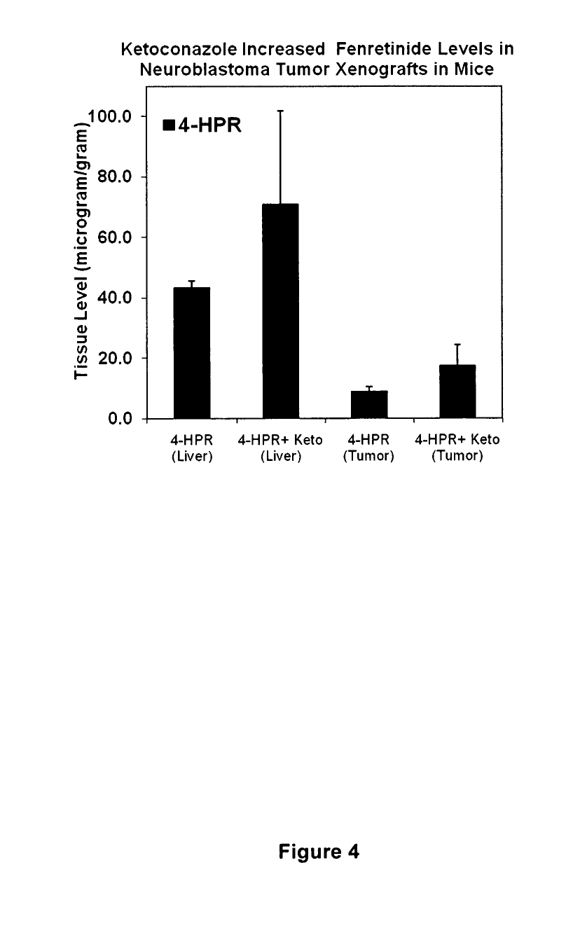

Ketoconazole increased fenretinide levels in human tumor xenograft tissue grown in nude mice. See FIG. 4. The human neuroblastoma cancer cell line, SMS-KCNR, was grown as a subcutaneous tumor xenograft in immunocompromised nude mice. Mice (n=2) bearing tumors (−150 mm³) were treated with fenretinide (i.e., N-(4-hydrophenyl)retinamide, 4-HPR) formulated for oral delivery in a vegetable oil (fatty acid) Lym-X-Sorb® matrix delivered by gavageat 180 mg/kg/day, divided into two daily doses, for five doses total (Control), or alternatively, mice (n=3) were treated with ketoconazole, 50 mg/kg/day, divided into two daily doses, for nine total doses, starting two days prior to fenretinide at 180 mg/kg/day, divided into two daily doses, for five doses total (4-HPR+KETO). Animals were sacrificed four hours after the last drug dose and fenretinide measured in liver and tumor tissue by a HPLC methodology. Results showed that ketoconazole increased fenretinide levels in both normal liver and tumor tissues. These data indicate that ketoconazole has the potential to increase fenretinide levels in tumor tissues and, thereby, increase fenretinide anti-tumor effects, over a treatment course.

### Example 5

### Effect of Other Inhibitors on Fenretinide Plasma Levels

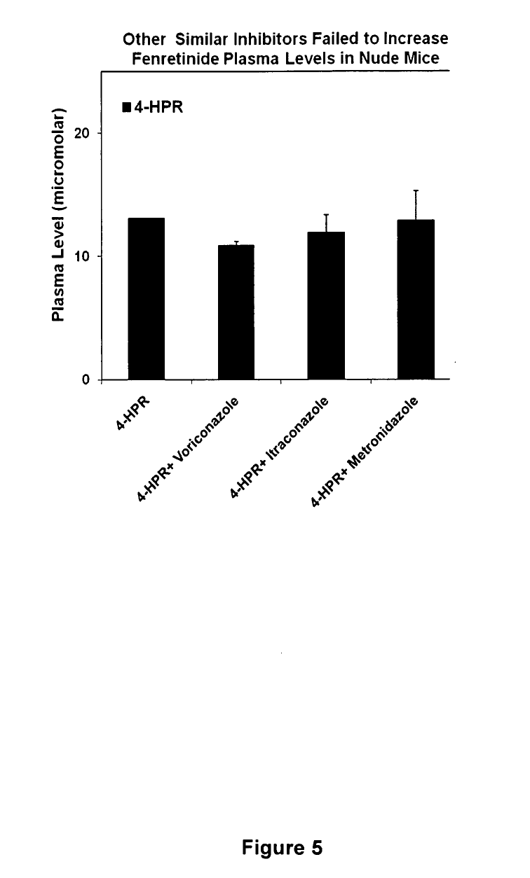

Other inhibitors did not increase fenretinide plasma levels. See FIG. 5. Fenretinide (i.e., N-(4-hydrophenyl)retinamide, 4-HPR) formulated for oral delivery in a vegetable oil (fatty acid) Lym-X-Sorb® matrix was delivered by gavage to nude (nu/nu) mice (n=3, per cohort) at 180 mg/kg/day, divided into two daily doses, for five doses total (Control), or voriconazole, at a loading dose of 200 mg/kg/day, divided into two daily doses, ×1 day, followed by 100 mg/kg/day, divided into two daily doses, for seven further doses, starting two days prior to fenretinide at 180 mg/kg/day divided, into two daily doses, for five doses total (4-HPR+Voriconazole); or itraconazole, at 60 mg/kg/day, divided into two daily doses, for nine doses, starting two days prior to fenretinide at 180 mg/kg/day, divided into two daily doses, for five doses total (4-HPR+Itraconazole); or metronidazole, at 360 mg/kg/day, divided into two daily doses, for nine doses, starting two days prior to fenretinide at 180 mg/kg/day, divided into two daily doses, for five doses total (4-HPR+Metronidazole). Voriconazole and itraconazole are imidazole antifungal antibiotics. Metronidazole is a nitroimidazole antibiotic. The human equivalent doses used for all three of these inhibitors was within the standard dose ranges for human treatment. Animals were sacrificed four hours after the last drug dosing and levels of fenretinide measured in the plasma by a HPLC methodology. None of these inhibitors increased fenretinide plasma levels (P=0.13, P=0.17, and P=0.23, respectively, one-sided t-test). These data demonstrate that ketoconazole has a singular and beneficial effect on fenretinide plasma and normal and tumor tissue levels compared to similar drugs (see Examples 2 and 4).

Those skilled in the art will recognize, or be able to ascertain using no more than routine experimentation many equivalents of the specific embodiments of the present invention herein disclosed. Such equivalents are intended to be encompassed by the following claims.

1. A method of increasing in the body an amount of a treatment agent, said method comprising administering an inhibitor of the metabolic degradation or conversion of the treatment agent to a subject undergoing treatment for a hyperproliferative disorder with said treatment agent.

2. The method of claim 1, wherein the treatment agent is a retinoid.

3. The method of claim 2, wherein the treatment agent is fenretinide or a fenretinide-derivative.

4. (canceled)

5. The method of claim 1, wherein the inhibitor is an inhibitor of hepatic or nonhepatic cytochrome P₄₅₀ enzymes.

6. The method of claim 5, wherein the inhibitor is an imidazole.

7. The method of claim 6, wherein the inhibitor is ketoconazole or triazole.

8. (canceled)

9. The method of claim 7, wherein the inhibitor is fluconazole.

10. The method of claim 1, wherein the inhibitor is an inhibitor of methyltransferases.

11. The method of claim 1, wherein the amount of the treatment agent is increased in the blood.

12. The method of claim 1 further comprising administering the treatment agent.

13. The method of claim 1, wherein the inhibitor is given before, during or after administration of the treatment agent.

14. The method of claim 1, wherein the treatment agent and the inhibitor are pharmaceutically compounded together for delivery.

15. The method of claim 1, wherein the treatment agent and the inhibitor are pharmaceutically compounded separately for delivery.

16. The method of claim 1, wherein the treatment agent is fenretinide and the inhibitor is ketoconazole.

17. The method of claim 1, wherein the treatment agent is fenretinide and the inhibitor is fluconazole.

18. The method of claim 1, wherein the hyperproliferative disorder is a cancer or tumor.

19. The method of claim 18, wherein the cancer is a cancer of the lung, breast, prostate, esophagus, stomach, colon, liver, pancreas, kidney, rectum, ovary, cervix, uterus, skin, brain, bone, bladder, head and neck, soft tissues, a leukemia or a lymphoma.

20. The method of claim 18, wherein the cancer or tumor is a cancer or tumor that has been shown to be the subject of a treatment effect by fenretinide or a fenretinide-derivative.

21. A method of treating a cancer or tumor comprising administering an inhibitor of the metabolic degradation or conversion of a treatment agent intended to treat the cancer or tumor, wherein said inhibitor increases the amount of the intended treatment agent in the body.

22. The method of claim 21, wherein the treatment agent is a retinoid.

23. The method of claim 22, wherein the treatment agent is fenretinide or a fenretinide-derivative.

24. (canceled)

25. The method of claim 21, wherein the inhibitor is an inhibitor of hepatic or nonhepatic cytochrome P₄₅₀ enzymes.

26. The method of claim 21, wherein the inhibitor is an inhibitor of methyltransferases.

27. The method of claim 21, wherein the hyperproliferative disorder is a cancer or tumor.

28. A method of increasing in a cancer or a tumor the amount of an agent intended to treat the cancer or tumor comprising administering to a subject in need of treatment (a) the intended treatment agent, and (b) an inhibitor of the metabolic degradation or conversion of the intended treatment agent.

29. The method of claim 28, wherein the treatment agent is a retinoid.

30. The method of claim 29, wherein the treatment agent is fenretinide or a fenretinide-derivative.

31. (canceled)

32. The method of claim 28, wherein the inhibitor is an inhibitor of hepatic or non-hepatic cytochrome P₄₅₀ enzymes.

33. The method of claim 28, wherein the inhibitor is an inhibitor of methyltransferases.

34. The method of claim 28, wherein the hyperproliferative disorder is a cancer or tumor.
